(19) United States
(12) Patent Application Publication
(10) Pub. No.: US 2009/0262344 A1
(43) Pub. Date: Oct. 22, 2009

(54) Measuring Equipment for quality of water

(76) Inventors: Sung Jin Yoon, Gyeonggi-do (KR); nam-il Kim, Chungbuk (KR); Han-Oh Park, Daejeon (KR)

Correspondence Address: CLARK & BRODY, 1090 VERMONT AVENUE, NW, SUITE 250, WASHINGTON, DC, 20005, US

(21) Appl. No.: 12/310,970

(22) PCT Filed: Jul. 4, 2008
(86) PCT No.: PCT/KR2008/003123

§ 371 Date: Mar. 13, 2009

(30) Foreign Application Priority Data
Jun. 8, 2007 (KR) 10-2007-0056324

(51) Int. Cl. G01N 33/18

(52) U.S. Cl. 356/317

(57) ABSTRACT

The present invention relates to equipment for measuring water quality using a fish or a water flea, an more particularly, to equipment for measuring water quality in which small organisms such as killifish (or medaka) or water flea are put into test chamber having a plurality of divided sections and behavior of the fishes or the water fleas accommodated in the test chamber is observed while continuously flowing water of a river or a water zone to be examined through the test chamber, thereby finding out and measuring existence of toxic element or harmful substance included in the water to be examined.

### TECHNICAL FIELD

The present invention relates to equipment for measuring water quality using fish or water flea, more particularly, to equipment for measuring water quality in which small organisms such as killifish (or medaka) or water flea are put into test chamber having a plurality of divided sections and behavior of the fish or the water flea introduced in the test chamber is observed while continuously flowing water of a river or a water zone to be examined through the test chamber, thereby finding out and measuring existence of toxic element or harmful substance included in the water to be examined.

### BACKGROUND ART

In general, a system for monitoring (monitoring and alarming) using a organisms having an ability of accurately and sensitively responding to specific chemical substance as a detector (test organism) in order to detect toxic element or harmful substance included in water that flows through a river is called as a bio-monitoring system or a early warning system.

The bio-monitoring system has been globally used for a long time since proper selection of the life to be used as the detector provides advantages of faster speed, simpler structure and superior detecting and measuring performance compared to a chemical sensor. A water flea, one of Crustacea, is generally used as the detector. The principle is as follows: regular activity (movement pattern) of the water flea is enlarged proportionally with increase in concentration of the toxic element and this results in variation in scattered and transmitted amounts of light that transmits the water containing the water flea therein, the variation being converted into a value of a measuring equipment and becoming a barometer of the measurement of water quality. In recent, in addition to the water flea, some organisms such as algae and small fish are also used as the detector. The fish has low accuracy but can be easily handled.

However, since the behavior of the detector is influenced by not only environmental factor such as the toxic element, etc. but also various internal factors such as health status or other instinct desires of the detector, a causal relationship between behavior pattern of the detector and measurement of water pollution or harmful substance is not much strong in some cases. Therefore, misjudgment occurs frequently in the aforementioned prior manner. Also, it is a challenge to qualitatively analyze sensitivity of the detector according to a range condition of the concentration of the pollutant and a range condition of measuring time.

### DISCLOSURE

### Technical Problem

An object of the present invention is to provide equipment for measuring water quality that can minimize influences of various internal factors such as health status or other instinct desires of the detector in an operation of bio-monitoring system and thus raise accuracy and reliability of the measurement of the water quality.

Another object of the present invention is to provide equipment for measuring water quality that can measure efficiently and accurately the water quality using fish, i.e. small killifish (or medaka) as detector in an efficient operation of bio-monitoring system.

Further object of the present invention is to provide equipment for measuring water quality that can measure efficiently and accurately the water quality using water flea as detector in an efficient operation of a bio-monitoring system.

### Technical Solution

In an aspect of the present invention, there is provided equipment for measuring water quality using detector, which includes test chamber provided with a plurality of containing spaces each containing the detector separately; a light source installed at a floor of the test chamber; heater for regulating temperature of water in the test chamber; a camera installed with a distance from the test chamber and monitoring and tracking behavior of the detector in the test chamber; a transferring unit for transferring behavior information of the detector taken by the camera; and a display unit for displaying the behavior information of the detector received from the transferring unit.

Preferably, the test chamber has a partition wall installed between adjacent containing spaces and separating the adjacent containing spaces, water in the test chamber being capable of passing through the partition wall and the test chamber is provided in plural sets for continuity of the measuring operation.

Preferably, the light source is a LED.

The detector (test organism) may be killifish (or medaka) or water flea.

Preferably, the partition wall of the test chamber has a plurality of holes having a size smaller than the size of the test organism when killifish (or medaka) is used as the detector, and the test chamber is a mesh net with a plurality of holes having a size smaller than the size of the test organism when the water flea is used as the detector.

### ADVANTAGEOUS EFFECTS

In the equipment for measuring water quality according to the present invention, influences of various internal factors such as health status or other instinct desires of the detector is minimized in an operation of a bio-monitoring system and thus reliability of the measurement of the water quality is raised.

Also, in the equipment for measuring water quality according to the present invention, small fish such as killifish (or medaka) or water flea having properties most similar to physiological properties of human body is used as a detector while monitoring is performed in a state that only one individual is contained in one containing space, and therefore efficiency and accuracy of the detecting operation can be enhanced.

### DESCRIPTION OF DRAWINGS

The above and other objects, features and advantages of the present invention will become apparent from the following description of preferred embodiments given in conjunction with the accompanying drawings, in which:

FIG. 1 is a conceptual view illustrating equipment for measuring water quality in a case of using a killifish (or a medaka) as a detector according to an embodiment of the present invention.

FIG. 2 is a perspective view illustrating the equipment for measuring water quality in a case of using a killifish (or a medaka) as a detector according to an embodiment of the present invention.

FIG. 3 is a conceptual view illustrating equipment for measuring water quality in a case of using a water flea as a detector according to an embodiment of the present invention.

FIG. 4 is a perspective view illustrating the equipment for measuring water quality in a case of using a water flea as a detector according to an embodiment of the present invention.

### DETAILED DESCRIPTION OF MAIN ELEMENTS

| | |
|---|---|
| 10, 30: | test chamber |
| 11, 31: | containing space |
| 12, 32: | partition wall |
| 13, 17: | water supplying passage |
| 14, 34: | flow gauge |
| 15, 16, 36: | valve |
| 18, 38: | water supplying part |
| 19, 39: | heater |
| 20, 40: | water supplying unit |
| 21, 41: | submergible pump |
| 23, 43: | water tank |

### Best Mode

Hereinafter, preferred embodiments of the present invention will be described in detail with reference to accompanying drawings.

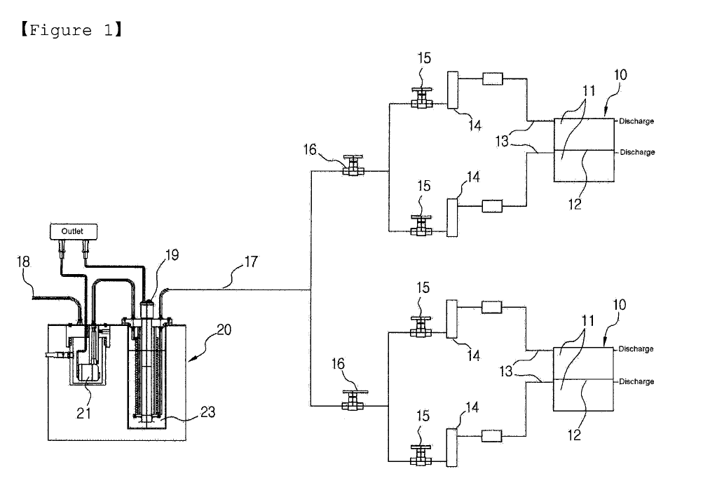

FIG. 1 is a conceptual view illustrating equipment for measuring water quality in a case of using a killifish (or a medaka) as a detector according to an embodiment of the present invention. As shown, there are provided two sets of test chamber 10 and each set has containing spaces 11 to contain a detector therein, the containing spaces 11 being divided into four. A partition wall 12 is formed between the containing spaces 11 of the test chamber 10 and the partition wall 12 is formed with rectangular holes (not shown) having a size smaller than the size of the killifish so that water in the test chamber can freely flow through the partition wall 12 but the killifish (or medaka) contained in the respective containing spaces 11 as the detector are not mixed. In other words, it is enough that the rectangular hole has a size not allowing the killifish (or medaka) to pass through the rectangular hole. Only one killifish (or medaka) is put in and contained in each containing space 11 of the test chamber 10. The reason why the test chamber 10 is divided into many containing spaces 11 and one containing space 11 contains only one killifish (or medaka) is because the detectors may interact with each other (e.g. fighting each other or producing fry) when a plurality of detectors is contained in a single space 11 and thus it is not only difficult to measure water quality but also harmful to consistency of measuring operation and result.

Meanwhile, a light source 51 is installed on the floor of the test chamber 10 to provide a day and night condition as same as the nature. It is preferable to use, as the light source, a LED that can obtain sufficient illuminance with small electric power and easily control brightness so as to establish the condition similar to day, night and dawn.

Also, the test chamber 10 is continuously supplied with water through a supplying part 18 of a water supplying unit 20 from water source of which water quality is to be measured and it is preferable to use a water tank 23 and a submergible pump 21 for continuity of the measurement. Of course, a heater 19 may be provided at a side of the water supplying unit 20 in order to control water temperature.

The water supplied from the water source is supplied via the water supplying unit 20 to the test chamber 10 through a water supplying passage 17, and the water supplying passage 17 is provided with valves such as needle valve 16 or ball valve 15 for opening and closing the water supplying passage 17 at suitable positions thereof, a flow gauge 14 for finely regulating amount of supplied water and a separate water discharge port (not shown).

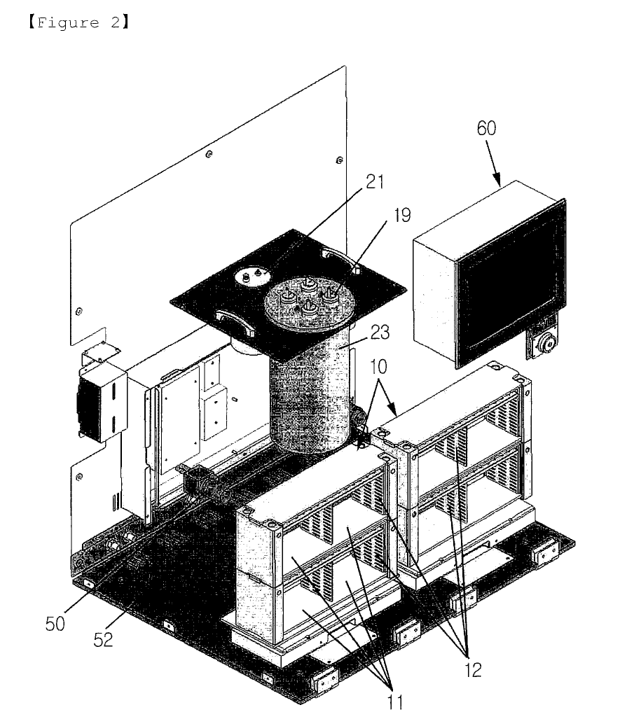

FIG. 2 is a perspective view illustrating the equipment for measuring water quality in a case of using a killifish (or a medaka) as a detector according to an embodiment of the present invention. The test chamber 10 is constituted of two sets and fixedly mounted on a substrate 52. Each test chamber 10 is provided with four containing spaces 11 and the partition walls 12 are installed between respective containing spaces 11. Each containing space 11 of the test chamber 10 contains only one killifish (or medaka) and behavior of the killifish (or medaka) is tracked and monitored by a camera 50 installed at a side surface of the test chamber 10. And, the behavior information of the killifish (or medaka), i.e. the detector taken by the camera 50 is transferred to a computer 60 through cable or wireless and the behavior information may displayed on a monitor of the computer. The behavior information of the detector is obtained as an image and is subjected to an image processing process using the computer. If the toxic element or harmful substance is contained in the supplied water, the detector that is a life exhibits abnormal action. From, this abnormal action, it is possible to detect inflow of the toxic element or harmful substance using the image processing algorithm of the computer.

In FIG. 2, the heater 19 is inserted in the water tank 23 located at an upper side of the test chamber 10 and the water of which temperature is regulated by the heater 19 is supplied to the test chamber 10 by the submergible pump 21.

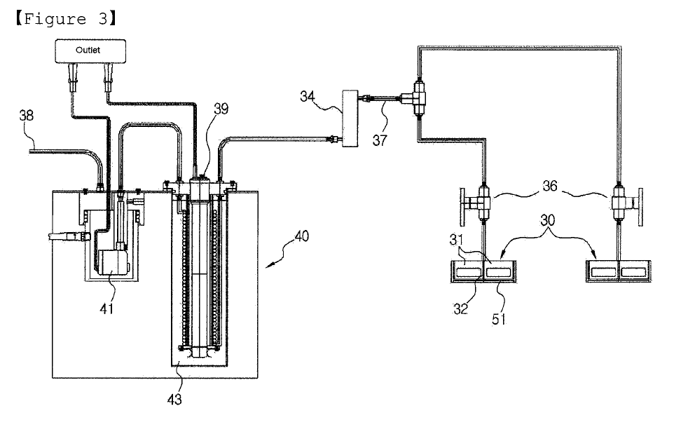

FIG. 3 is a conceptual view illustrating equipment for measuring water quality in a case of using water flea as a detector according to an embodiment of the present invention. Describing in the range not overlapped with FIGS. 1 and 2, there are provided, as shown, two sets of test chamber 30 and each set has containing spaces 31 to contain a detector therein, the containing spaces 31 being divided into four. A partition wall 32 is formed between the containing spaces 31 of the test chamber 30 and the partition wall 32 is formed of a mesh net (not shown) having a hole size smaller than the size of the water flea so that water in the test chamber can freely flow through the partition wall 32 but the water fleas contained in the respective containing spaces 31 as the detector are not mixed. In other words, it is enough that the mesh hole has a size not allowing the water flea to pass through the mesh hole. Only one water flea is put in and contained in each containing space 31 of the test chamber 30. The reason why the test chamber 30 is divided into many containing spaces 31 and one containing space 31 contains only one water flea is because the detectors may interact with each other when a plurality of detectors is contained in a single space 31 and thus it is not only difficult to measure water quality but also harmful to consistency of measuring operation and result.

Meanwhile, a light source 51 is installed on the floor of the test chamber 30 to provide a day and night condition as same as the nature. It is preferable to use, as the light source, a LED that can obtain sufficient illuminance with small electric power and easily control brightness so as to establish the condition similar to day, night and dawn.

Also, the test chamber 30 is continuously supplied with water through a supplying part 38 of a water supplying unit 40 from water source of which water quality is to be measured and it is preferable to use a water tank 43 and a submergible pump 41 for continuity of the measurement. Of course, a heater 39 may be provided at a side of the water supplying unit 40 in order to control water temperature.

The water supplied from the water source is supplied via the water supplying unit 40 to the test chamber 30 through a water supplying passage 37, and the water supplying passage 37 is provided with valves such as needle valve 36 for opening and closing the water supplying passage 37 at suitable positions thereof and a flow gauge 34 for finely regulating amount of supplied water.

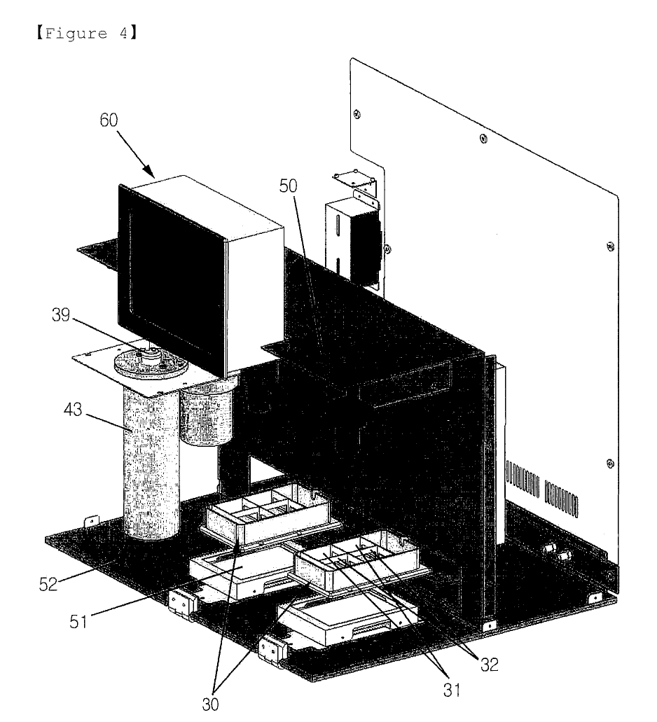

FIG. 4 is a perspective view illustrating the equipment for measuring water quality in a case of using a water flea as a detector according to an embodiment of the present invention. The test chamber 30 is constituted of two sets and fixedly mounted on a substrate 52. Each test chamber 30 is provided with four containing spaces 31 and the partition walls 32 are installed between respective containing spaces 31. Each containing space 31 of the test chamber 30 contains only one water flea and behavior of the water flea is taken and monitored by a camera 50 installed at a side surface of the test chamber 30. And, the behavior information of the water flea, i.e. the detector taken by the camera 50 is transferred to a computer 60 through cable or wireless and the behavior information may displayed on a monitor of the computer. In FIG. 4, the heater 39 is inserted in the water tank 43 located at an upper side of the test chamber 30 and the water of which temperature is regulated by the heater 39 is supplied to the test chamber 30 by a submergible pump (not shown).

Although specific embodiments of the present invention have been described for illustrative purposes, those skilled in the art will appreciate that various modifications, additions and substitutions are possible, without departing from the scope and the spirit of the invention as disclosed in the accompanying claims.

### INDUSTRIAL APPLICABILITY

According to the equipment for measuring water quality of the present invention, influences of various internal factors such as health status or other instinct desires of the detector is minimized in an operation of a bio-monitoring system and thus reliability of the measurement of the water quality is raised. Also, small organisms such as killifish (or medaka) or water flea having properties most similar to physiological properties of human body are used as detector while monitoring is performed in a state that only one individual is contained in one containing space, and therefore efficiency and accuracy of the detecting operation is enhanced.

1. Equipment for measuring water quality using a detector, comprising:
test chamber (10, 30) provided with a plurality of containing spaces (11, 31) each containing the detector separately;
a light source (51) installed at a floor of the test chamber (10, 30);
heater (19, 39) for regulating temperature of water in the test chamber (10, 30);
a camera (50) installed with a distance from the test chamber (10, 30) and monitoring and tracking behavior of the detector in the test chamber (10, 30);
a transferring unit (not shown) transferring for behavior information of the detector taken by the camera (50); and
a display unit (60) for displaying the behavior information of the detector received from the transferring unit.

2. The equipment for measuring water quality using a detector as set forth in claim 1, wherein the test chamber (10, 30) has a partition wall (12, 32) installed between adjacent containing spaces (11, 31) and separating the adjacent containing spaces (11, 31), water in the test chamber (10, 30) being capable of passing through the partition wall (12, 32).

3. The equipment for measuring water quality using a detector as set forth in claim 2, wherein the test chamber (10, 30) is provided in plural sets.

4. The equipment for measuring water quality using a detector as set forth in claim 2, wherein the light source (51) is a LED.

5. The equipment for measuring water quality using a detector as set forth in claim 2, wherein the detector is killifish (or medaka).

6. The equipment for measuring water quality using a detector as set forth in claim 5, wherein the partition wall (12) of the test chamber has a plurality of holes (not shown) having a size smaller than the size of the killifish (or medaka).

7. The equipment for measuring water quality using a detector as set forth in claim 2, wherein the detector is a water flea.

8. The equipment for measuring water quality using a detector as set forth in claim 7, wherein the partition wall (12) of the test chamber is a mesh net with a plurality of holes having a size smaller than the size of the water flea.
